(19) United States
(12) Patent Application Publication
(10) Pub. No.: US 2016/0131624 A1
(43) Pub. Date: May 12, 2016

(54) CARBON DIOXIDE GROUND LEAKAGE MONITORING SYSTEM

(71) Applicant: CHUGAI TECHNOS CORPORATION, Hiroshima-shi, Hiroshima (JP)

(72) Inventors: Yoshikazu Naito, Hiroshima-shi, Hiroshima (JP); Satoshi Fukuma, Hiroshima-shi, Hiroshima (JP); Taro Kawamura, Hiroshima-shi, Hiroshima (JP); Yuki Nakamura, Hiroshima-shi, Hiroshima (JP)

(73) Assignee: CHUGAI TECHNOS CORPORATION, Hiroshima-shi, Hiroshima (JP)

(21) Appl. No.: 14/772,065

(22) PCT Filed: Mar. 20, 2014
(86) PCT No.: PCT/JP2014/057713

§ 371 Date: Sep. 1, 2015

(51) Int. Cl. G01N 33/00

(52) U.S. Cl. CPC G01N 33/004; G01N 33/0006

(57) ABSTRACT

Provided is a carbon dioxide ground leakage monitoring system that is low cost, capable of real-time multi-point monitoring on a wide scale, and capable of providing the public with easy-to-understand information.

A plurality of monitoring posts 100 and a field data server 200 and the field data server 200 and an administrative server 300 are connected by communication lines in respective sections therebetween. The plurality of monitoring posts 100 measure a concentration of carbon dioxide having leaked above the ground, and transmits the measured carbon dioxide data to the field data server 200, the field data server 200 transmits the data transmitted from the plurality of monitoring posts 100 to the administrative server 300, and the administrative server 300 discloses carbon dioxide ground leakage information on the Internet based on the carbon dioxide data transmitted from the field data server 200.

### TECHNICAL FIELD

The present invention relates to a carbon dioxide ground leakage monitoring system capable of sensing that carbon dioxide has reached a high concentration on the surface of the ground due to leakage if by chance leakage occurs to cause a high carbon dioxide concentration in a facility and area, etc., where carbon dioxide emitted from a thermal power station or the like is captured and stored underground.

### BACKGROUND ART

Conventionally, carbon dioxide emissions that are major contributors to global warming have been a major problem, and reductions in emissions have been required internationally. On the other hand, as a carbon dioxide disposal technology, a technology for capturing carbon dioxide emitted from a thermal power station or the like and storing the same underground has been drawing attention. Using this technology for containing carbon dioxide underground leads to a reduction in the concentration of carbon dioxide in the atmosphere.

Such capture and storage of carbon dioxide emitted from a thermal power station or the like has been called “CCS (Carbon dioxide Capture and Storage) ,” and a facility and area therefor has been called a “CCS site.”

In a storage process of carbon dioxide in a CCS project, carbon dioxide captured from a thermal power station or the like is transported a few tens of kilometers through pipelines, and then sealed in a storage layer lying a few thousands of meters underground. In the CCS site, carbon dioxide stored underground should not leak to the surface of the ground again, but the possibility of leakage due to various reasons such as unexpected crustal change and aging degradation of the buried pipelines cannot be disclaimed.

Therefore, for example, Patent Literature 1 discloses an invention in which a carbon dioxide concentration sensing device is buried in an unsaturated zone under the land surface of a site where carbon dioxide is stored in the ground to measure a carbon dioxide concentration, the measured carbon dioxide concentration is transmitted by a communication device, and the transmitted carbon dioxide concentration is compared with a reference carbon dioxide concentration by a time period by a monitoring server to output a normality signal or an abnormality signal.

### CITATION LIST

### Patent Literature

Patent Literature 1

Japanese Published Unexamined Patent Application No. 2011-64671

Also in Enhanced Oil Recovery (EOR), which is a technology of injecting a gas or chemical into an oil layer and mixing the same with crude oil under high pressure to improve the fluidity of crude oil within the oil layer for enhancing recovery of oil resources, carbon dioxide is used as an injection gas in some cases, and there is likewise a possibility of ground leakage of carbon dioxide.

### SUMMARY OF INVENTION

### Technical Problem

Meanwhile, it is difficult to identify the amount (concentration, duration of leakage) and the location of carbon dioxide leaking from the transportation pipelines of carbon dioxide spanning a few tens of kilometers or the storage layer lying a few thousands of meters underground, and real-time multi-point monitoring is necessary on a considerably wide scale also including surrounding residential areas and surrounding country (such as grasslands).

However, conventional carbon dioxide concentration measuring devices have been used mainly indoors or in enclosed spaces and used for securing comfort and ensuring the quality of agricultural and industrial products, and have not been suitable for outdoor uses.

Also, devices for measuring carbon dioxide concentrations in the ground or on the surface of the ground outdoors include one that sucks air from the ground by a container having a built-in pump to perform a measurement, one that performs a measurement when carbon dioxide having exited the ground passes through the inside of a container having opening portions at its top and bottom, and one that is provided with an openable cover at an upper portion of a container and automatically opens and closes the cover according to the situation, however, the high-functioning measuring devices have been high in cost and also consume a large amount of power, and have also had a high risk of malfunction. On the other hand, low-functioning measuring devices could not perform measurement under the effects of rain, wind, snow, condensation, etc., in some cases. Therefore, the conventional measuring devices have not been suitable for long-term continuous and real-time use in an outdoor environment.

Also, in order to perform real-time multi-point monitoring on a wide scale, it has been necessary to build enormous dedicated infrastructures such as electricity and communication lines for the conventional measuring devices, and enormous operational and maintenance costs have also been necessary for these dedicated infrastructures after introduction.

Thus, it has been difficult to realize a monitoring system capable of real-time multi-point monitoring at low cost and on a wide scale using the conventional carbon dioxide concentration measuring device.

Also, while leakage monitoring in a CCS project has currently been carried out for the purpose of operations management and academic use by its project implementer, for validating the effectiveness of the CCS project and promoting dissemination, it is an important challenge to obtain social consensus from the public, and a monitoring system that is familiar and easy to understand by the public and not through project implementer's eyes is necessary. However, there has been no monitoring system that allows public access to the monitoring status so as to allow anyone in the world to simply confirm safety.

The present invention has been made to solve the conventional problems described above, and it is an object to provide a carbon dioxide ground leakage monitoring system that is low cost, capable of real-time multi-point monitoring on a wide scale, and capable of providing the public with easy-to-understand information.

### Solution to Problem

In order to solve the above-described problems, a carbon dioxide ground leakage monitoring system of the present invention includes a plurality of monitoring posts, a field data server, and an administrative server, and in the carbon dioxide ground leakage monitoring system, the plurality of monitoring posts and the field data server and the field data server and the administrative server are connected by communication lines in respective sections therebetween, the plurality of monitoring posts measure a concentration of carbon dioxide having leaked above the ground, and transmits the measured carbon dioxide data to the field data server, the field data server transmits the data transmitted from the plurality of monitoring posts to the administrative server, and the administrative server discloses carbon dioxide ground leakage information on the Internet based on the carbon dioxide data transmitted from the field data server.

And preferably, the communication lines that connect the plurality of monitoring posts and the field data server are a mesh-type wireless communication network having the plurality of monitoring posts as nodes.

And preferably, the plurality of monitoring posts measure a concentration of carbon dioxide having leaked above the ground by an interval operation.

And preferably, the plurality of monitoring posts monitor a remaining charge level of a battery equipped therein, and change the interval operation in cycle according to the remaining charge level of the battery.

And preferably, the field data server instructs the plurality of monitoring posts to change the interval operation in cycle.

And preferably, a carbon dioxide concentration measurement section of the plurality of monitoring posts is composed of a retention chamber, a carbon dioxide concentration measuring sensor, and a protective cover, the retention chamber is a cylindrical structure opened at its top and bottom, and is installed with its lower open portion closely fitted to soil, the carbon dioxide concentration measuring sensor is disposed inside the retention chamber, and the protective cover is installed so as to surround a periphery of the retention chamber, and is provided in an upper portion thereof with a vent.

And preferably, the field data server instructs the plurality of monitoring posts to calibrate the carbon dioxide concentration measuring sensor.

### Advantageous Effects of Invention

The carbon dioxide ground leakage monitoring system according to the present invention includes a plurality of monitoring posts, a field data server, and an administrative server, and the plurality of monitoring posts and the field data server and the field data server and the administrative server are connected by communication lines in respective sections therebetween. Moreover, the carbon dioxide ground leakage monitoring system measures the concentration of carbon dioxide having leaked above the ground and transmits the measured carbon dioxide data to the field data server by the plurality of monitoring posts, and transmits the data transmitted from the plurality of monitoring posts to the administrative server by the field data server. Accordingly, disposing the plurality of monitoring posts in the CCS site enables real-time multi-point monitoring on a wide scale. Also, the carbon dioxide ground leakage monitoring system discloses carbon dioxide ground leakage information on the Internet by the administrative server based on the carbon dioxide data transmitted from the field data server. Accordingly, easy-to-understand information can be provided to the public.

Also, providing the communication lines that connect the plurality of monitoring posts and the field data server as a mesh-type wireless communication network having the plurality of monitoring posts as nodes allows performing operations with reduced transmitting power using near-field wireless transmission.

Also, the plurality of monitoring posts measuring the concentration of carbon dioxide having leaked above the ground by an interval operation allows performing operations with the power consumption suppressed.

Also, the plurality of monitoring posts monitoring the remaining charge level of a battery equipped therein and changing the interval operation in cycle according to the remaining charge level of the battery allows performing long-term stable operations.

Also, the field data server instructing the plurality of monitoring posts to change the interval operation in cycle allows performing operations with the power consumption suppressed.

Also, the carbon dioxide concentration measurement sections of the plurality of monitoring posts are each composed of the retention chamber, the carbon dioxide concentration measuring sensor, and the protective cover. Moreover, the retention chamber is a cylindrical structure opened at the top and bottom and is installed with its lower open portion closely fitted to the soil, the carbon dioxide concentration measuring sensor is disposed inside the retention chamber, and therefore, the concentration of carbon dioxide having leaked from the soil and retained in the retention chamber can be measured by the carbon dioxide concentration measuring sensor. Further, the protective cover is installed so as to surround a periphery of the retention chamber and is provided in an upper portion thereof with the vent, which can thus make the carbon dioxide concentration in the retention chamber less likely to receive the effects of wind, rain, snow, etc., and also allow appropriately emitting carbon dioxide through the vent such that carbon dioxide is not excessively retained inside the retaining chamber.

Also, the field data server instructing the plurality of monitoring posts to calibrate the carbon dioxide concentration measuring sensor allows performing a more accurate measurement.

As above, the present invention can provide a carbon dioxide ground leakage monitoring system that is low cost, capable of real-time multi-point monitoring on a wide scale, and capable of providing easy-to-understand information to the public.

### BRIEF DESCRIPTION OF DRAWINGS

FIG. 1 is a configuration diagram of a carbon dioxide ground leakage monitoring system according to an embodiment of the present invention.

FIG. 2 is a configuration view of a monitoring post.

FIG. 3 is a configuration diagram of a field data server.

### DESCRIPTION OF EMBODIMENTS

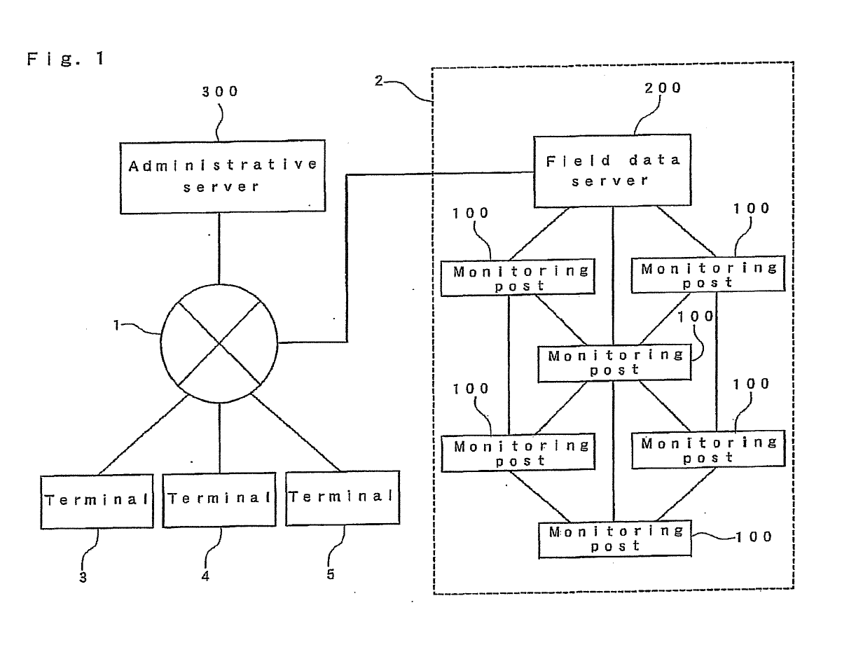

Now, a carbon dioxide ground leakage monitoring system according to an embodiment of the present invention will be described with reference to FIG. 1 to FIG. 3. First, referring to FIG. 1, description will be given of an overall configuration of the carbon dioxide ground leakage monitoring system according to the present embodiment. Although the present embodiment relates to a carbon dioxide ground leakage monitoring system in a CCS project, the present invention can be applied, also to Enhanced Oil Recovery (EOR) that uses carbon dioxide as an injection gas, likewise as a carbon dioxide ground leakage monitoring system.

As shown in FIG. 1, the carbon dioxide groundleakage monitoring system according to the present embodiment is composed of a plurality of monitoring posts 100, a field data server 200, and an administrative server 300.

The plurality of monitoring posts 100 and the field data server 200 are disposed in a CCS site 2. The monitoring post 100 serves a function of measuring a carbon dioxide concentration at a spot where the same is disposed and transmitting the measured data to the field data server 200. In, order to monitor ground leakage of carbon dioxide on a wide scale, the monitoring posts 100 are disposed in large numbers in regions along transportation pipelines from a thermal power station or the like and regions having carbon dioxide storage layers in the ground. Particularly, the disposition is prioritized in a range where the possibility of leakage is high (near transportation pipelines, around an injection well, etc.). The field data server 200 serves a function of collecting in a batch data from the plurality of monitoring posts 100 and transmitting the same to the administrative server 300, and is disposed at, for example, a local office in the CCS site.

The administrative server 300 serves a function of disclosing carbon dioxide ground leakage information on the Internet based on the carbon dioxide data transmitted from the field data server 200, and is disposed at, for example, headquarters or the like of a CCS project implementer.

The plurality of monitoring posts 100 and the field data server 200 and the field data server 200 and the administrative server 300 are connected by communication lines in respective sections therebetween.

Of the communication lines, ones that connect the plurality of monitoring posts 100 and the field data server 200 are preferably provided as a mesh-type wireless communication network having the plurality of monitoring posts 100 as nodes. Moreover, multihop communications by a near-field wireless module are preferably performed to perform operations with reduced transmitting power.

Also, the field data server 200 and the administrative server 300 are preferably connected by way of the Internet. In the present embodiment, the field data server 200 and the administrative server 300 are respectively connected to the Internet 1. Connecting by way of the Internet allows easily transmitting and receiving data even when the headquarters of the CCS project implementer and the CCS site are distant from each other. In addition, a configuration may be adopted in which the field data server 200 and the administrative server 300 are connected by a dedicated line or the like therebetween.

In FIG. 1, reference signs 3, 4, and 5 respectively denote terminals which are connectable to the Internet, and which allow browsing over the Internet carbon dioxide ground leakage information disclosed by the administrative server 300. As users of the terminals 3, 4, and 5, the public, a third-party organization, the CCS project implementer, etc., are assumed.

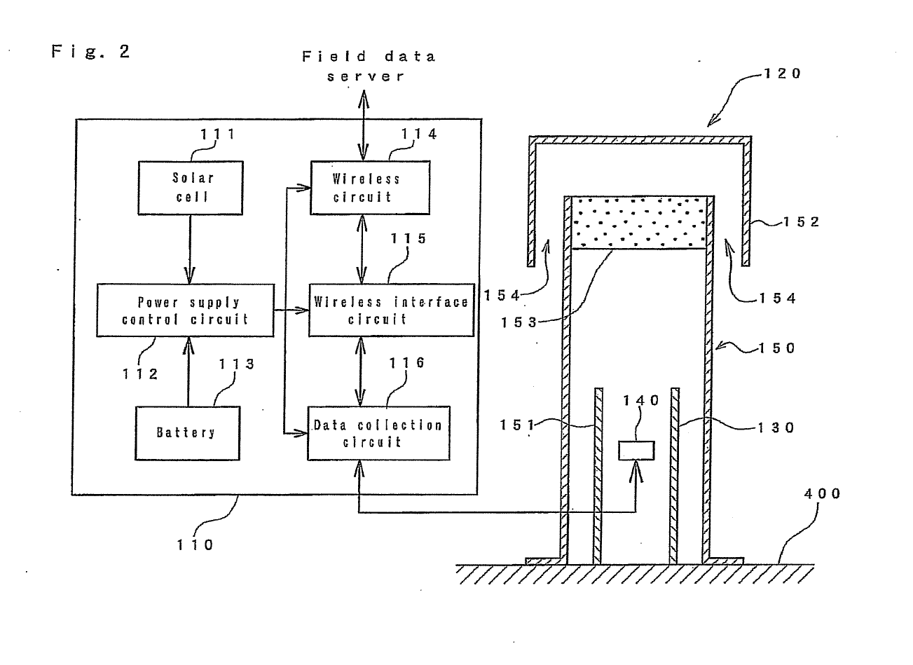

Next, referring to FIG. 2, description will be given of a configuration of the monitoring post 100. The monitoring post 100 is composed of an electrical equipment section 110 and a measurement section 120. The electrical equipment section 110 manages the supply of electricity of the monitoring post 100 as a whole and collects data measured by the measurement section 120 and transmits the data to the field data server 200. The measurement section 120 is installed on soil 400 of a measurement point, and measures the concentration of carbon dioxide that leaks from the soil 400. The electrical equipment section 110 and the measurement section 120 may be connected by a cable as separate structures, or may be provided as an integral structure.

In the electrical equipment section 110, a solar cell 111, a power supply control circuit 112 that manages the supply of electricity, and a battery 113 are provided. The power supply control circuit 112 controls electricity from the solar cell 111 and the battery 113 to manage the supply of electricity of the monitoring post 100 as a whole. Providing a power source as a standalone power supply system consisting of the solar cell 111 and the battery 113 enables operations without preparing dedicated infrastructures.

Also, in the electrical equipment section 110, a wireless circuit 114, a wireless interface circuit 115, and a data collection circuit 116 are provided. The wireless circuit 114 performs transmission and reception of data with the field data server 200. Measurement data collected by the data collection circuit 116 is transmitted from the wireless circuit 114 via the wireless interface circuit 115.

The measurement section 120 is composed of a retention chamber 130, a carbon dioxide concentration measuring sensor 140, and a protective cover 150.

The retention chamber 130 is for retaining carbon dioxide leaked from the soil 400, is a cylindrical structure opened at the top and bottom, and is installed with its lower open portion closely fitted to the soil 400. In an interior of the retention chamber 130, the carbon dioxide concentration measuring sensor 140 is disposed. A supply of electricity to the carbon dioxide concentration measuring sensor 140 is performed from the electrical equipment section 110. In addition, sensors that measure the temperature or humidity may be provided inside the retention chamber 130, in addition to the carbon dioxide concentration measuring sensor 140. The retention chamber 130 is installed for the purpose of securing a channel when carbon dioxide diffuses from soil into air to suppress diffusive convection so as to slow (delay) the behavior of the carbon dioxide concentration.

The protective cover 150 is composed of a main body portion 151 that is cylindrical and has a structure opened at the top and bottom, a cap portion 152 that covers an upper portion of the main body portion 151, and a windshield member 153 provided in the upper open portion of the main body portion 151. The main body portion 151 is in a cylindrical shape larger than that of the retention chamber 130, and is installed outside the retention chamber 130 with its lower open portion closely fitted to the soil 400. Also, between the upper portion of the main body portion 151 and the cap portion 152, a vent 154 is formed. Based on the above, the protective cover 150 is installed so as to surround a periphery of the retention chamber 130, and is provided in an upper portion thereof with the vent 154. Moreover, the protective cover 150 can make the carbon dioxide concentration in the retention chamber 130 less likely to receive the effects of wind, rain, snow, etc.

The windshield member 153 is provided such that carbon dioxide retained in the retention chamber 130 does not receive the effect of external wind, and suffices by using porous urethane foam or the like. In addition, the windshield structure is not limited to the above, and suffices with one for which a complicated channel structure is provided, for example, such as to resist wind while allowing diffusive ventilation from the interior.

Based on the above, carbon dioxide having leaked from the soil 400 is retained in the retention chamber 130, and then passes through the windshield member 153 from the upper portion of the protective cover 150 to be externally emitted by way of the vent 154.

In addition, the measurement section 120 in the present embodiment is not provided with a pump, a movable mechanism, and the like, thus has a reduced risk of failure as well as ease of maintenance, and is therefore capable of a stable and continuous measurement.

As in the present embodiment, in the standalone power supply system using unstable sunlight, power saving of the system is important in order to ensure economy and safety.

Therefore, the function of the electrical equipment 110 is preferably realized by a low-power-consumption microcomputer and a near-field wireless module that is used in the field of HEMS and BEMS. Also, the carbon dioxide concentration measuring sensor 140 preferably adopts an NDIR method that is low in power consumption. Collection and wireless transmission of data can thereby be performed with a power consumption on the order of a few milli-watts.

Also, the monitoring posts 100 may measure the concentration of carbon dioxide having leaked above the ground by an interval operation. For example, operating the near-field wireless module and the carbon dioxide concentration measuring sensor 140 by an interval operation (operation to automatically repeat “power-on” to “running” to “power-off”) allows further suppressing the power consumption.

Also, the remaining charge level of a battery equipped therein may be monitored so as to change the interval operation in cycle according to the remaining charge level of the battery. A long-term stable operation is thereby enabled.

In addition, because of the interval operation of the carbon dioxide concentration measuring sensor 140, there is a problem that a measurement of the carbon dioxide concentration is disabled every constant cycle, but this problem can be solved by retaining leaked carbon dioxide in the retention chamber 130 to slow (delay) the behavior of the carbon dioxide concentration.

The monitoring posts 100 are wirelessly connected and not provided by a method of burying in the ground, and therefore, relocation and addition thereof is relatively easy.

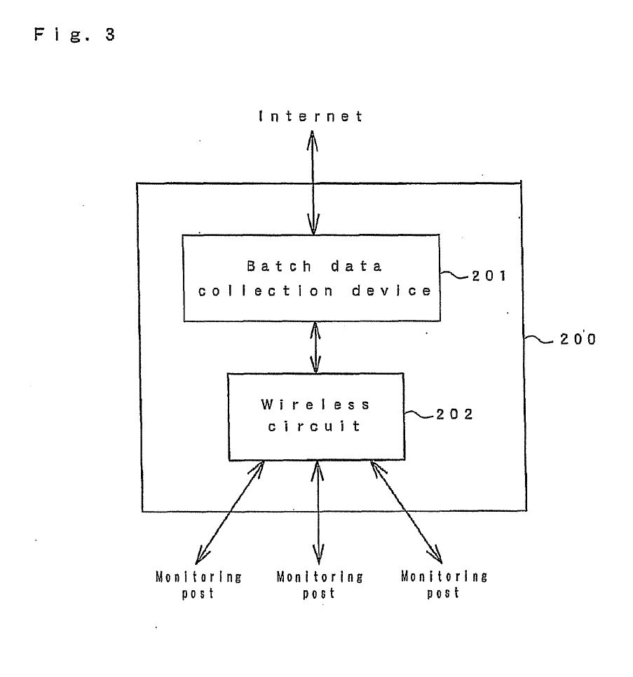

Next, referring to FIG. 3, description will be given of a configuration of the field data server 200. The field data server 200 is composed of a batch data collection device 201 and a wireless circuit 202.

The batch data collection device 201 consists of a CPU (such as an embedded PC) for collecting data from the respective monitoring posts 100. The wireless circuit 202 is for performing wireless communications with the respective monitoring posts 100, and is preferably realized by a near-field wireless module that is used in the field of HEMS and BEMS.

The field data server 200, in a mesh-type wireless communication network having the plurality of monitoring posts 100 as nodes, functions as a sink node, and collects in a batch data (carbon dioxide concentrations, temperatures, humidities, remaining battery charge levels, solar power generation capacities, radio field intensities, etc.) of the plurality of monitoring posts 100 connected thereto by wireless communication.

Also, the field data server 200 maybe able to perform control, for the plurality of monitoring posts 100 connected thereto, such as instructing to change the interval operation in cycle and calling internal data by remote control. Because control such as setting changes can be performed in a batch by remote control, a huge amount of work does not occur on site even when the monitoring posts 100 are installed at a large number of spots on a wide scale.

Also, the field data server 200 may be able to instruct the plurality of monitoring posts 100 connected thereto to calibrate the carbon dioxide concentration measuring sensor 140 by remote control.

The field data server 200 is connected to the Internet 1, and transmits necessary data to the administrative server 300 via the Internet 1. Because connections to the Internet 1 are concentrated in one place in the present system, communication costs can be held down. In addition, the field data server 200 maybe made accessible from an external personal computer by an SSH connection or the like so as to allow control from a further remote place.

The administrative server 300 is connected to the Internet 1, and receives data from the field data server 200 via the Internet 1. Moreover, the administrative server 300 discloses carbon dioxide ground leakage information on the Internet based on the transmitted carbon dioxide data. Information disclosure to the public can thereby be performed to develop a better understanding for the CCS project.

In addition, if a concentration exceeding a preset value is detected, the information may be immediately sent by e-mail or the like to the CCS project implementer and the like.

The data to be disclosed on the Internet can be provided, by a processing such as mapping real-time measurement data on a map, as one that is easy to understand for the public. Also, a viewer may be allowed to freely switch measurement data in display format (graphs, numerical values, accumulated values, daily, annular, etc.) for viewing.

The carbon dioxide ground leakage monitoring system according to the present embodiment includes the plurality of monitoring posts 100, the field data server 200, and the administrative server 300, and the plurality of monitoring posts 100 and the field data server 200 and the field data server 200 and the administrative server 300 are connected by communication lines in respective sections therebetween. Moreover, the carbon dioxide ground leakage monitoring system measures the concentration of carbon dioxide having leaked above the ground and transmits the measured carbon dioxide data to the field data server 200 by the plurality of monitoring posts 100, and transmits the data transmitted from the plurality of monitoring posts 100 to the administrative server 300 by the field data server 200. Accordingly, disposing the plurality of monitoring posts 100 in the CCS site 2 enables real-time multi-point monitoring on a wide scale. Also, the carbon dioxide ground leakage monitoring system discloses carbon dioxide ground leakage information on the Internet by the administrative server 300 based on the carbon dioxide data transmitted from the field data server 200. Accordingly, easy-to-understand information can be provided to the public

Also, providing the communication lines that connect the plurality of monitoring posts 100 and the field data server 200 as a mesh-type wireless communication network having the plurality of monitoring posts 100 as nodes allows performing operations with reduced transmitting power using near-field wireless transmission.

Also, the plurality of monitoring posts 100 measuring the concentration of carbon dioxide having leaked above the ground by an interval operation allows performing operations with the power consumption suppressed.

Also, the plurality of monitoring posts 100 monitoring the remaining charge level of a battery equipped therein and changing the interval operation in cycle according to the remaining charge level of the battery allows performing long-term stable operations.

Also, the field data server 200 instructing the plurality of monitoring posts 100 to change the interval operation in cycle allows performing operations with the power consumption suppressed.

Also, the carbon dioxide concentration measurement sections 120 of the plurality of monitoring posts 100 are each composed of the retention chamber 130, the carbon dioxide concentration measuring sensor 140, and the protective cover 150. Moreover, the retention chamber 130 is a cylindrical structure opened at the top and bottom and is installed with its lower open portion closely fitted to the soil 400, the carbon dioxide concentration measuring sensor 140 is disposed inside the retention chamber 130, and therefore, the concentration of carbon dioxide having leaked from the soil 400 and retained in the retention chamber 130 can be measured by the carbon dioxide concentration measuring sensor 140. Further, the protective cover 150 is installed so as to surround a periphery of the retention chamber 130 and is provided in an upper portion thereof with the vent 154, which can thus make the carbon dioxide concentration in the retention chamber 130 less likely to receive the effects of wind, rain, snow, etc., and also allow appropriately emitting carbon dioxide through the vent 154 such that carbon dioxide is not excessively retained inside the retaining chamber 130.

Also, the field data server 200 instructing the plurality of monitoring posts 100 to calibrate the carbon dioxide concentration measuring sensor 140 allows performing a more accurate measurement.

As above, the present invention can provide a carbon dioxide ground leakage monitoring system that is low cost, capable of real-time multi-point monitoring on a wide scale, and capable of providing easy-to-understand information to the public.

While the carbon dioxide ground leakage monitoring system according to an embodiment of the present invention has been described above, the present invention is not limited to the foregoing embodiment, and various other modifications can be made. For example, regarding the disposition of the administrative server 300, there may be an arrangement of disposing not in a remote place but in the same position as that of the field data server 200.

### REFERENCE SINGS LIST

1 Internet
2 CCS site
3 Terminal
4 Terminal
5 Terminal
100 Monitoring post
110 Electrical equipment section
111 Solar cell
112 Power supply control circuit
113 Battery
114 Wireless circuit
115 Wireless interface circuit
116 Data collection circuit
120 Measurement section
130 Retention chamber
140 Carbon dioxide concentration measuring sensor
150 Protective cover
151 Main body portion
152 Cap portion
153 Windshield member
154 Vent
200 Field data server
201 Batch data collection device
2.02 Wireless circuit
300 Administrative server

1. A carbon dioxide ground leakage monitoring system including a plurality of monitoring posts, a field data server, and an administrative server, wherein
the plurality of monitoring posts and the field data server and the field data server and the administrative server are connected by communication lines in respective sections therebetween,
the plurality of monitoring posts measure a concentration of carbon dioxide having leaked above the ground, and transmits the measured carbon dioxide data to the field data server,
the field data server transmits the data transmitted from the plurality of monitoring posts to the administrative server, and
the administrative server discloses carbon dioxide ground leakage information on the Internet based on the carbon dioxide data transmitted from the field data server.

2. The carbon dioxide ground leakage monitoring system according to claim 1, wherein the communication lines that connect the plurality of monitoring posts and the field data server are a mesh-type wireless communication network having the plurality of monitoring posts as nodes.

3. The carbon dioxide ground leakage monitoring system according to claim 1, wherein the plurality of monitoring posts measure a concentration of carbon dioxide having leaked above the ground by an interval operation.

4. The carbon dioxide ground leakage monitoring system according to claim 3, wherein the plurality of monitoring posts monitor a remaining charge level of a battery equipped therein, and change the interval operation in cycle according to the remaining charge level of the battery.

5. The carbon dioxide ground leakage monitoring system according to claim 3, wherein the field data server instructs the plurality of monitoring posts to change the interval operation in cycle.

6. The carbon dioxide ground leakage monitoring system according to claim 1, wherein a carbon dioxide concentration measurement section of the plurality of monitoring posts is composed of a retention chamber, a carbon dioxide concentration measuring sensor, and a protective cover,
the retention chamber is a cylindrical structure opened at its top and bottom, and is installed with its lower open portion closely fitted to soil,
the carbon dioxide concentration measuring sensor is disposed inside the retention chamber, and
the protective cover is installed so as to surround a periphery of the retention chamber, and is provided in an upper portion thereof with a vent.

7. The carbon dioxide ground leakage monitoring system according to claim 6, wherein the field data server instructs the plurality of monitoring posts to calibrate the carbon dioxide concentration measuring sensor.
